(19) United States
(12) Patent Application Publication
(10) Pub. No.: US 2011/0306568 A1
(43) Pub. Date: Dec. 15, 2011

(54) COMPOSITION CONTAINING GLUCOSYLGLYCEROL

(76) Inventors: Thomas Schwarz, Mannheim (DE); Julia Klein, Bochum (DE)

(21) Appl. No.: 13/123,805

(22) PCT Filed: Oct. 28, 2009
(86) PCT No.: PCT/EP09/07704

§ 371 Date: Aug. 31, 2011

(30) Foreign Application Priority Data
Oct. 28, 2008 (DE) 10 2008 053 549.4

(51) Int. Cl. A01N 43/16; A61Q 19/00; B32B 5/16; A01P 1/00; C09K 3/20; A23L 1/236; A61K 8/60; A01P 3/00

(52) U.S. Cl. 514/25; 252/182.29; 426/548; 428/402.2

(57) ABSTRACT

The invention relates to a composition containing 20 to 80% w/w of glucosyl-glycerol and 0.5 to 20% w/w of one or several mono- and/or disaccharides. Surprisingly, it has been found that an especially aqueous solution containing a high concentration of glucosylglycerol offers exceptionally high long-term stability. The composition has bactericidal and fungicidal effects.

The invention relates to a composition on the basis of glucosylglycerol as well as the use of this composition.

Glucosylglycerol, or more specific 2-O-α-D-glucosylglycerol, is a natural substance synthetized, for example, from cyanobacteria which make use of its properties for osmoprotective purposes. In this manner cyanobacteria are capable of growing in saline media with concentrations of up to 1.5 M NaCl. The molecule accumulates in high concentrations in the cytoplasm and in this way causes the existing osmotic pressure existing in such an environment due to the high salt concentration to be reduced thus protecting the cell against water losses. An example here is the cyanobacterium Synechocystis sp. PCC 6803.

Furthermore, the molecule is also produced by plants of genus myrothamnus. These plants are growing in humid-to-dry environments. Myrothamnus flabellifolia is a small shrub found in the southern region of Africa growing on rock slabs up to a height of 60 cm. The plant survives completely unharmed and in desiccated condition drought periods occurring in the southern African region and lasting several months. However, as soon as it rains again the plant begins to sprout within a few hours so that it is also known under the byword of “resurrection plant”. The structure of 2-O-α-D-glucosylglycerol is as follows:

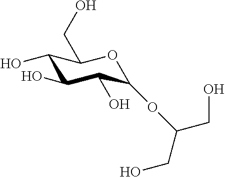

DE 195 40 749 A1, for example, describes the use of glucosyl glycerides such as glucosylglycerol for cosmetic and dermatological preparations. The substance may, for example, be employed as moisturizer, i.e. as a substance having moisture-adding properties. Aside from this, glucosylglycerol has previously been described in the context of its use in foodstuff, as stabilizer of biomolecules and microorganisms, as medical agent for the therapy of cancer or as antibacterial agent.

Glucosylglycerol can be produced both chemically and enzymatically. The synthesis of natural 2-O-α-D-glucosylglycerol in pure form is in the context of stereochemistry practically unfeasible by chemical methods since only insignificant yields are obtainable in this manner. Although a microbial synthesis could be realized yields obtainable in this way were found to be low. An enzymatic synthesis utilizing the transglucosylation by means of α-glucosidase has a major disadvantage in that the regioselectivity of the process is wrong because the enzyme prefers the glucosylation of the primary instead of the secondary hydroxyl group of the glycerol. Accordingly, when using α-glucosidase the product mixture merely contains 30% of the correct natural glucosylglycerol so that the product can only be isolated with much effort and expenditure.

WO 2008/034158 A2 discloses an especially preferable method for the production of 2-O-α-D-glucosylglycerol. In this case a saccharose phosphorylase is allowed to interact with a blend that has a glucosyl donor and glycerol as glucosyl acceptor. Preferably, the glucosyl donor is saccharose. Aside from this glucose 1-phosphate and α-D-glucose 1-fluoride can be used. Selectivity and yield (>95%) are excellent.

The conversion of saccharose with glycerol in the presence of saccharose phosphorylase at first results in the formation of a glucosyl enzyme intermediate causing fructose to be liberated. Subsequently, the glucosyl enzyme intermediate is attacked by a nucleophile, in this case glycerol, so that the desired 2-O-α-D-glucosylglycerol is produced and the enzyme is again liberated. As a side reaction the hydrolysis of the glucosyl enzyme intermediate takes place causing glucose to be formed. Cleaning of the product is then achieved by chromatography.

It has been found, however, that after the product had been cleaned by column chromatography sugars of certain amount, in particular of the mono- and disaccharides glucose, fructose and saccharose still remained in the product. This turned out to be a problem due to the fact that the sugars still left in the product could have served as nutrient for bacteria or fungi thus resulting in the shelf life of the glucosylglycerol composition to be reduced. On the other hand, the addition of preservation agents, bactericides or fungicides has to be avoided to the extent possible in view of the product's application in cosmetics or as food additive.

It is therefore the objective of the invention to provide a composition based on glucosylglycerol that is not prone to increased bacteria growth and thus offers a longer shelf life.

According to the invention this objective is reached by providing a composition that contains 20 to 80% w/w of glucosylglycerol and 0.5 to 20% w/w of one or several mono- and/or disaccharides. The composition is preferably provided in the form of an aqueous solution.

Surprisingly, it has been found that a composition containing glucosylglycerol of an increased concentration ranging between 20 and 80% w/w offers long-term stability and is not affected by increased bacteria growth. Despite the presence of mono- and/or disaccharides the growth of bacteria or fungi does not increase, on the contrary the number of colony-forming units even reduces significantly as a result of the composition provided in accordance with the invention.

The glucosylglycerol employed is preferably the naturally occurring 2-O-α-D glucosylglycerol which for example is accumulated by cyanobacteria of genus Synechocystis. Basically, a comparable effect can also be expected, however, from the β-glycosidic linkage of glucose to the glycerol molecule or from the linkage of glucose to glycerol at the 1-position. It is therefore conceivable as well to use also 1-O-α glucosylglycerol, 1-O-β glucosylglycerol and 2-O-β glucosylglycerol in D and L configuration in addition to natural glucosylglycerol.

The surprisingly bactericide effects of the inventive composition can be seen from the following table showing viable count results of E. coli DSM 1576 with aqueous solutions of various concentrations of 2-O-α-D glucosylglycerol as well as 5% w/w glucose having been used in the investigation:

| Glucosylglycerol | Colony-forming units (CFU/g) | | | | | |
|---|---|---|---|---|---|---|
| (% w/w) | 0 days | 2 days | 7 days | 14 days | 21 days | 28 days |
| 50 | 300,000 | 150,000 | <1,000 | <100 | <100 | <100 |
| 30 | 300,000 | 130,000 | <1,000 | <100 | <100 | <100 |
| 20 | 300,000 | 280,000 | 100,000 | 5,000 | <100 | <100 |
| 0 | 300,000 | 4,000,000 | 2,200,000 | 1,500,000 | 1,100,000 | 1,000,000 |

CFU = colony-forming unit

As is evident from the above table using a high glucosylglycerol concentration results in the number of colony-forming units to be significantly reduced within a few days whereas the number of bacteria colonies initially rises steeply when a reference solution (5% w/w of glucose) is used. Obviously, glucosylglycerol of high concentration thus has highly bactericidal effects.

In addition, a another test was analogously conducted with mold Aspergillus niger and yeast fungi Candida albicans , with an aqueous solution containing 50% w/w of 2-O-α-D glucosylglycerol and 3.5% w/w of glucose being used in this case.

| | Colony-forming units (CFU/g) | | | | | |
|---|---|---|---|---|---|---|
| | 0 days | 2 days | 7 days | 14 days | 21 days | 28 days |
| A. niger | 750,000 | 350,000 | 200,000 | 170,000 | 160,000 | 12,000 |
| C. albicans | 750,000 | 320,000 | 200,000 | 190,000 | 120,000 | 45,000 |

Glucosylglycerol of high concentration thus has not only bactericidal but also fungicidal effects so that the invention also relates to the use of the concentrated glucosylglycerol composition as bactericide or fungicide.

Preferably, the glucosylglycerol in the composition is used at concentrations ranging between 30 and 70, also preferred at concentrations between 40 and 60 and especially preferred at approximately 50% w/w. A 50-% solution offers excellent long-term stability.

The concentration of mono- and/or disaccharides typically ranges between 1 and 10% w/w, in particular between 3 and 7% w/w. In the course of the above described synthesis fructose obtained as a result of the enzymatic conversion, glucose as side product as well as saccharose as educt are produced primarily.

The aqueous solution is a clear, colorless to light yellow liquid having a pH value of between 5.5 and 6.0. Aside from the substances mentioned further substances may be present, in particular glycerin which was not converted completely.

The composition according to the invention may be used as a constituent of skin care agents, for example moisturizers. The composition may also be used as a component of sweetening agents for diabetics, dietary supplements or to stabilize biomolecules such as proteins and lipids.

To suit the relevant application the composition may also contain further substances, for example cosmetic auxiliary substances such as e.g. carrier agents, perfumes, solutizers, vitamins, stabilizers, anti-foaming substances, thickeners, colorants, surfactants, emulsifiers or moisturizing substances. Basically conceivable is also the addition of preservation agents and bactericides, even if a substantial long-term stability is ensured without additives of such nature.

The inventive composition may also be used in the form of microcapsules. The microencapsulation of liquids is a method sufficiently known to those having expertise for example in the field of pharmacy. Making use of this method can facilitate an oral administration, improve the tolerability and, if need be, also cause the release to be appropriately delayed.

1. Composition containing 20 to 80% w/w of glucosylglycerol and 0.5 to 20% w/w of one or several mono- and/or disaccharides.

2. Composition according to claim 1, characterized in that the composition is an aqueous solution.

3. Composition according to claim 1, characterized in that the glucosylglycerol is 2-O-α-D glucosylglycerol.

4. Composition according to claim 1, characterized in that the concentration of glucosylglycerol ranges between 30 and 70% w/w.

5. Composition according to claim 4, characterized in that the concentration of glucosylglycerol ranges between 40 and 60% w/w, and in particular is approximately 50 w/w.

6. Composition according to claim 1, characterized in that the concentration of mono- and/or disaccharides ranges between 1 and 10% w/w, in particular between 3 and 7% w/w.

7. Composition according to claim 1, characterized in that the mono- or disaccharides are glucose, fructose and/or saccharose.

8. Use of a composition according to claim 1 as an ingredient of a skin care agent.

9. Use of a composition according to claim 1 for the stabilization of biomolecules.

10. Use of a composition according to claim 1 as an ingredient of a sweetening agent.

11. Use of a composition containing between 20 and 80% w/w of glucosylglycerol, in particular 2-O-α-D glucosylglycerol, as bactericide.

12. Use of a composition containing between 20 and 80% w/w of glucosylglycerol, in particular 2-O-α-D glucosylglycerol, as fungicide.

13. Microcapsules containing a composition according to claim 1.
